(19) United States
(12) Patent Application Publication
(10) Pub. No.: US 2010/0280326 A1
(43) Pub. Date: Nov. 4, 2010

(54) SURGICAL DEVICES FOR INTERNAL ACCESS THROUGH TISSUE

(76) Inventors: Christopher J. Hess, Cincinnati, OH (US); Rudolph H. Nobis, Mason, OH (US); Andrew M. Zwolinski, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/475,012

(22) Filed: May 29, 2009

Related U.S. Application Data
(63) Continuation-in-part of application No. 12/435,075, filed on May 4, 2009.

(51) Int. Cl. A61B 1/32

(52) U.S. Cl. 600/206

(57) ABSTRACT

Methods and devices are provided for providing access through tissue to a surgical site. In one embodiment, a surgical access device can be configured to move between a first, expanded configuration, and a second, compressed configuration. As the device moves from the first configuration to the second configuration, a longitudinal length of the device can decrease, thereby allowing the device to be securely positioned in tissue and reducing a distance that the device extends into a body cavity.

### RELATED APPLICATION

This continuation in part application incorporates by reference and claims priority to U.S. patent application Ser. No. 12/435,075 filed May 4, 2009 in the names of Nobis et al.

### FIELD OF THE INVENTION

The present invention relates to surgical devices for providing access through tissue to a surgical site.

### BACKGROUND OF THE INVENTION

Access ports are widely used in medical procedures to gain access to anatomical cavities ranging in size from the abdomen to small blood vessels, such as veins and arteries, epidural, pleural and subarachnoid spaces, heart ventricles, and spinal and synovial cavities. The use of access ports has become more common as they provide minimally invasive techniques for establishing a portal for a number of procedures, such as those involving the abdominal cavity. Reduced postoperative recovery time, markedly decreased post-operative pain and wound infection, and improved cosmetic outcome are well established benefits of minimally invasive surgery, derived mainly from the ability of surgeons to perform an operation utilizing smaller incisions of the body cavity wall.

In many surgical procedures, it is desirable to provide one or more working channels into a body cavity through which various instruments can be passed to view, engage, and/or treat tissue to achieve a diagnostic or therapeutic effect. In laparoscopic abdominal procedures for example, the abdominal cavity is generally insufflated with CO₂ gas to a pressure of around 15 mm Hg. The abdominal wall is pierced and one or more tubular cannulas, each defining a working channel, are inserted into the abdominal cavity. A laparoscopic telescope connected to an operating room monitor can be used to visualize the operative field and can be placed through one of the working channels. Other laparoscopic instruments such as graspers, dissectors, scissors, retractors, etc. can also be placed through one or more of the working channels to facilitate various manipulations by the surgeon and/or surgical assistant(s).

While effective, there can be disadvantages when using a typical access port. For example, the access port could extend a distance above and/or a distance below the tissue in which it is positioned, which can interfere with access to the surgical field. For another example, tissue thicknesses vary by patient, and a typical access port can have a size too large or too small for secure positioning within a patient's tissue. Moreover, it can be difficult and time-consuming during the stress of surgery to choose a properly sized access port, particularly in a single surgical procedure using multiple access ports positioned in differently sized tissue openings.

Accordingly, there is a need for improved methods and devices for providing access through tissue to a surgical site.

### SUMMARY OF THE INVENTION

The present invention generally provides methods and devices for providing access through tissue to a surgical site. In one embodiment, a surgical device is provided that includes a flexible retractor configured to be positioned in tissue to form an access pathway therethrough into a body cavity, and at least one member operatively associated with the flexible retractor and configured to automatically adjust a longitudinal length of the retractor to approximate a depth of a tissue opening within which the flexible retractor is positioned. The at least one member can have at least a first contracted configuration and a second extended configuration.

In one embodiment, the member may comprise one or more elongate members which extend both axially and circumferentially with respect to the flexible retractor. The elongate members may, in addition to providing adjustment of the length of the flexible tissue retractor to various depths (e.g. various thicknesses of abdominal wall structures), also provide circumferential load carrying capability, such as in the form of hoop loads. Without being limited by theory, the axially and circumferentially extending members may also assist in maintaining the flexible retractor in an open configuration against the skin/tissue forces which would otherwise tend to press against and possibly close or partially close the access opening through the retractor.

In one embodiment, the at least one member can comprise a plurality of interwoven elongate members which are disposed on a surface of or within a flexible membrane of the tissue retractor. The interwoven elongate members may be formed of a shape memory material, such as a shape memory metal alloy, such as an alloy having a temperature induced or stress induced phase transition. In one embodiment, a woven structure of Nitinol wire may be employed. Each elongate member may be have a monofilament or multiple filament (e.g. braided) structure.

In another embodiment, a tissue retractor may be provided with a non-metallic flexible membrane and an elastically extensible woven or non-woven network associated with the membrane of the retractor. The elastically extensible network can have an elongated configuration and one or more contracted configurations, such that the network resiliently retracts a distal portion of the retractor upward against an internal body surface of a body cavity when the tissue retractor is disposed in an incision.

The elongate members may be in the form of spring elements, or otherwise have spring like properties for resiliently biasing the retractor to a retracted configuration, while permitting the retractor to be extended to an extended configuration, such as when the retractor is being inserted through an incision opening.

The flexible retractor may be formed in a variety of ways. For instance, an axially and circumferentially extending interwoven or network structure having resilient biasing properties may be joined to a surface of the flexible membrane of retractor, or positioned between first and second layers of the retractor. Alternatively, a network structure may be formed integrally with the flexible membrane of retractor, such as by molding the membrane with an integral network structure.

A surgical device of the present invention may include a seal housing associated with a proximal end of the retractor. The seal housing may be releasably joined to the retractor, and the seal housing may have one or more seals associated with one or more instrument ports for accommodating surgical instruments inserted through the housing and the retractor into the body.

### BRIEF DESCRIPTION OF THE DRAWINGS

The invention will be more fully understood from the following detailed description taken in conjunction with the accompanying drawings, in which:

FIG. 1 is a perspective view of one embodiment of a surgical access device disclosed in the above incorporated U.S. patent application Ser. No. 12/435,075;

FIG. 2 is a partial cross-sectional view of an axial support member positioned between two films of a retractor as disclosed in the above incorporated U.S. patent application Ser. No. 12/435,075;

FIG. 3 is a side view of one embodiment of a surgical access device having a plurality of axial support members in a compressed configuration as disclosed in the above incorporated U.S. patent application Ser. No. 12/435,075;

FIG. 4 is a side view of the surgical access device of FIG. 3 with the plurality of axial support members in an expanded configuration as disclosed in the above incorporated U.S. patent application Ser. No. 12/435,075;

FIG. 5 is a side partially cross-sectional view of the surgical access device of FIG. 4 positioned in an opening in tissue disclosed in the above incorporated U.S. patent application Ser. No. 12/435,075;

FIG. 6 is a side partially cross-sectional view of the surgical access device of FIG. 5 positioned in an opening in tissue, moved to the compressed configuration of FIG. 3, and having a surgical instrument inserted therethrough;

FIG. 7 illustrates a surgical device according to one embodiment of the present application, depicting a surgical instrument having a seal housing disposed at a proximal end of a flexible retractor, and the flexible retractor including a member extending circumferentially and axially for biasing the retractor to a retracted configuration.

FIG. 8 illustrates the device of FIG. 7 with the flexible retractor extended to an extended configuration.

FIG. 9. Illustrates the device of FIG. 7 disposed in a patient's body such that the seal housing is disposed outside the body, the tissue retractor extends through a first, relatively thin layer of tissue, and the distal end of the retractor is disposed within a body cavity of the patient.

FIG. 10 illustrates the device of FIG. 7 with the retractor disposed in a second, relatively thicker layer of tissue.

FIG. 11 illustrates the device of FIG. 7 with the retractor disposed in a third, relatively thick layer of tissue.

FIG. 12 illustrates a portion of a flexible retractor according to one embodiment having a woven network of elongate members associated with the membrane of the tissue retractor.

FIG. 13 illustrates a portion of a flexible retractor according to another embodiment having a non-woven network associated with the membrane of the tissue retractor.

FIG. 14 illustrates a coil-like elongate member extending the full axial length of the retractor and circumferentially around the full circumference of the retractor.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for providing access through tissue to a surgical site. In one embodiment, a surgical access device can be configured to move between a first, expanded configuration, and a second, compressed configuration. As the device moves from the first configuration to the second configuration, a longitudinal length of the device can decrease, thereby allowing the device to be securely positioned in tissue and reducing a distance that the device extends into a body cavity.

The various surgical access devices described herein can generally be configured to allow one or more surgical instruments to be inserted therethrough through one or more independent sealing ports or access ports formed in a proximal housing, hereinafter generally referred to as a housing, of the device and into a body cavity. The sealing ports can each define working channels extending through the proximal housing and aligned with a distal retractor. The distal retractor, hereinafter generally referred to as a retractor, can be configured as a wound protector, or other member for forming a pathway through tissue. The retractor can extend from the proximal housing of the device, and it can be configured to be positioned within an opening in a patient's body, such as the umbilicus. Any and all of the surgical access devices described herein can also include various other features, such as one or more ventilation ports to allow evacuation of smoke during procedures that utilize cautery, and/or one or more insufflation ports through which the surgeon can insufflate the abdomen to cause pneumoperitenium, as described by way of non-limiting example in U.S. Patent Application No. 2006/0247673 entitled “Multi-port Laparoscopic Access Device” filed Nov. 2, 2006, which is hereby incorporated by reference in its entirety. The insufflation port can be located anywhere on the device, can have any size, and can accept a leur lock or a needle, as will be appreciated by those skilled in the art.

Any and all embodiments of a surgical access device can also include one or more safety shields positioned through, in, and around any of the components and/or tissue to protect the components against puncture or tear by surgical instruments being inserted through the device. Exemplary embodiments of safety shields are described in more detail in U.S. Patent Publication No. 2006/0247673 entitled “Multi-port Laparoscopic Access Device” filed Nov. 2, 2006, U.S. patent application Ser. No. 12/399,625 entitled “Methods and Devices for Providing Access to a Body Cavity” filed on Mar. 6, 2009, U.S. patent application Ser. No. 12/399,482 entitled “Methods and Devices for Providing Access to a Body Cavity” filed on Mar. 6, 2009, and U.S. patent application Ser. No. 12/242,765 entitled “Surgical Access Device” filed on Sep. 30, 2008, which are hereby incorporated by reference in their entireties.

In any and all of the surgical access device embodiments disclosed herein, an engagement and/or release mechanism can be included to allow certain components of the surgical access device to be removable as needed, such as removable coupling of a housing and a retractor. Any engagement and release mechanism known in the art, e.g., a snap-lock mechanism, corresponding threads, etc., can be used to releasably mate components of the device. Exemplary embodiments of an engagement and release mechanisms are described in more detail in previously mentioned U.S. patent application Ser. No. 12/242,765 entitled “Surgical Access Device” filed on Sep. 30, 2008, U.S. patent application Ser. No. 12/399,625 entitled “Methods and Devices for Providing Access to a Body Cavity” filed on Mar. 6, 2009, and U.S. patent application Ser. No. 12/399,482 entitled “Methods and Devices for Providing Access to a Body Cavity” filed on Mar. 6, 2009 and in U.S. Pat. No. 7,371,227 entitled “Trocar Seal Assembly,” issued May 13, 2008 and U.S. Pat. No. 5,628,732 entitled “Trocar With Improved Universal Seal,” issued May 13, 2007, which are hereby incorporated by reference in their entireties.

In use, as further discussed below, the surgical access devices disclosed herein can be used to provide access to a patient's body cavity. The device's retractor can be positionable within an opening in a patient's body such that a distal portion of the retractor extends into a patient's body cavity and a proximal portion configured to couple to the device's housing is positioned adjacent to the patient's skin on an exterior of the patient's body. A lumen in the retractor can form a pathway through the opening in a patient's body so that surgical instruments can be inserted from outside the body to an interior body cavity. The elasticity of the skin of the patient can assist in the retention of the retractor in the body opening or incision made in the body. Also as discussed further below, the retractor can be configured to automatically adjust its longitudinal length to correspond with a thickness of the patient's skin. The retractor can be placed in any opening within a patient's body, whether a natural orifice or an opening made by an incision. As a non-limiting example, the retractor can be placed through the umbilicus. In one embodiment, the retractor can be substantially flexible so that it can easily be maneuvered into and within tissue as needed. In other embodiments, the retractor can be substantially semi-rigid. The flexible membrane of the retractor can be formed of any suitable material known in the art, e.g., silicone, urethane, thermoplastic elastomer, and rubber.

Typically, during surgical procedures in a body cavity, such as the abdomen, insufflation is provided through the surgical access device to expand the body cavity to facilitate the surgical procedure. Thus, in order to maintain insufflation within the body cavity, most surgical access devices include at least one seal disposed therein to prevent air and/or gas from escaping when surgical instruments are inserted therethrough. Various sealing elements are known in the art, but typically the surgical access device can include at least one instrument seal that forms a seal around an instrument disposed therethrough, but otherwise does not form a seal when no instrument is disposed therethrough; at least one channel seal or zero-closure seal that seals the working channel created by the sealing port when no instrument is disposed therethrough; or a combination instrument seal and channel seal that is effective to both form a seal around an instrument disposed therethrough and to form a seal in the working channel when no instrument is disposed therethrough. A person skilled in the art will appreciate that various seals known in the art can be used including, e.g., duckbill seals, cone seals, flapper valves, gel seals, diaphragm seals, lip seals, iris seals, etc. A person skilled in the art will also appreciate that any combination of seals can be included in any of the embodiments described herein, whether or not the seal combinations are specifically discussed in the corresponding description of a particular embodiment. Exemplary embodiments of various seal protectors are described in more detail in U.S. Pat. No. 5,342,315 entitled “Trocar Seal/Protector Assemblies,” issued Aug. 30, 1994 and U.S. Pat. No. 7,163,525 entitled “Duckbill Seal Protector,” issued Jan. 16, 2007, which are hereby incorporated by reference in their entireties.

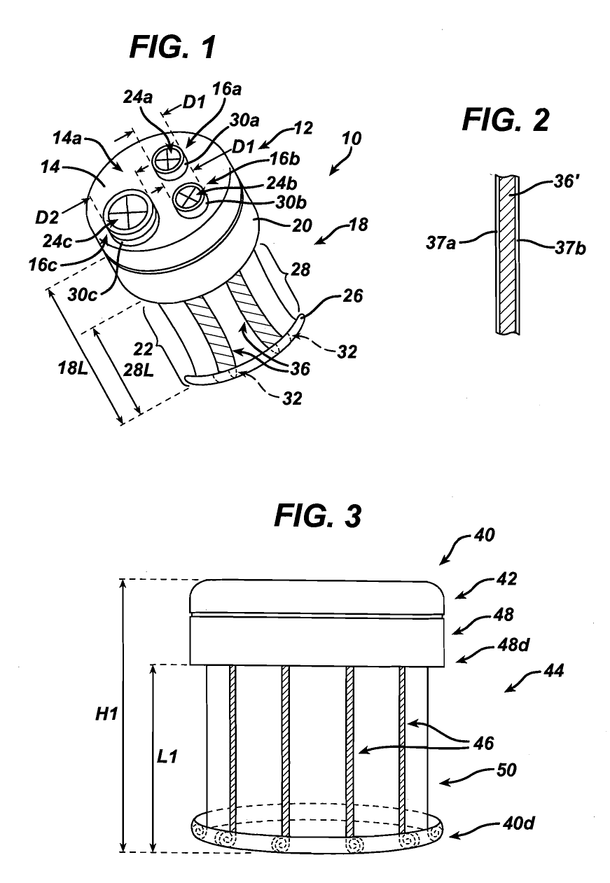

FIG. 1 shows a surgical access device 10 as described in the above incorporated U.S. patent application Ser. No. 12/435,075. The device 10 is provided having a housing 12 configured to have one or more surgical instruments inserted therethrough. Although the housing 12 can have any configuration, in this illustrated embodiment, the housing 12 includes a seal base 14 configured to support at least one sealing or access port, e.g., first, second, and third sealing ports 16a, 16b, 16c, and configured to form a seat and seal between the base 14 and a distal portion of the device 10, e.g., a retractor 18. The housing 12 can be fixedly or removably coupled to the retractor 18 configured to distally extend from the housing 12 and to provide a pathway through tissue into a body cavity. In this embodiment, the retractor 18 includes a proximal retractor portion or proximal retractor base 20 coupled to a distal retractor portion 22.

As noted above, the retractor 18 can extend distally from the housing 12, and it can be configured to be positioned in an opening formed in tissue. The retractor 18 can, as shown in this exemplary embodiment, include a substantially flexible distal portion 22 having a proximal flange (not shown) and a distal flange 26 with an inner elongate portion 28 extending therebetween. A retractor retaining band (not shown), e.g., an o-ring, can be positioned between the proximal retractor base 20 and the flexible distal portion 22 to help form a secure seal therebetween. The inner elongate portion 28 can have a diameter less than a diameter of the proximal flange and the distal flange 26, which can have the same diameter or different diameters from one another, and can be configured to be positioned within tissue. The proximal flange can be configured to be seated within the proximal retractor base 20 as illustrated in this embodiment, or the proximal retractor base 20 can be configured to be seated within the proximal flange. The proximal retractor base 20 can optionally be attached to the proximal flange using an adhesive, sealant, complementary threads, or any other attachment mechanism, as will be appreciated by a person skilled in the art. A proximal o-ring (not shown) can optionally be positioned within the proximal flange to help provide structural support to the retractor 18 if the proximal flange is seated within the proximal retractor base 20. The proximal o-ring can be substantially flexible or substantially rigid as needed for use in a particular application.

The retractor 18 can also include one or more axial support members 36 configured to automatically adjust a longitudinal length 18L of the retractor 18, e.g., by adjusting a longitudinal length 28L of the retractor's inner elongate portion 28, such that the retractor 18 can move between expanded and compressed configurations, as discussed further below. Although the retractor 18 in this illustrated embodiment includes four axial support members 36 (two are obscured), the retractor 18 can include any number of axial support members 36. Further, although the device 10 is shown with the axial support members 36 equidistantly spaced around a circumference or perimeter of the retractor 18 in the inner elongate portion 28, the device 10 can include any number of axial support members 36 arranged in any way on the retractor 18 or on the retractor 18 and the housing 12.

The axial support members 36 can have any size, shape, and configuration, same or different from any one or more of the other axial support members 36. The axial support member 36 can be flexible or rigid depending on the intended use. In one exemplary embodiment, the axial support members 36 can be semi-rigid to help provide structural integrity to the retractor 18. Any material can be used to form the axial support members 36, such as spring steel, e.g., 301 stainless steel, 302 stainless steel, 17-7 (313) stainless steel, or a shape memory material, e.g., Nitinol. In an exemplary embodiment, the axial support members 36 can include springs, such as constant force springs. Generally, constant force springs include an extension spring configured to provide a constant pressure profile and to have a first, expanded configuration in which the constant force spring can be uncoiled or substantially straight and a second, compressed configuration in which the constant force spring can be at least partially coiled with the constant force spring turning or wrapping on itself to form a tight coil. Any constant force spring can be used, such as type 301 stainless steel constant force extension springs available from McMaster-Carr Supply Company of Elmhurst, Ill.

The axial support members 36 can longitudinally extend along any portion of the retractor 18 (and optionally along any portion of the housing 12). As in the illustrated embodiment, the axial support members 36 can each continuously extend along the entire longitudinal length 28L of the inner elongate portion 28.

The axial support members 36 can be mated to the device 10 in any way. As in this illustrated embodiment, proximal ends (not shown) of the axial support members 36 can be mated to the proximal retractor base 20 and/or to the housing 12, and distal ends 32 of the axial support members 36 can be mated to the distal flange 26. The proximal ends and the distal ends 32 of the axial support members 36 can be mated to any one or more portions of the device 10 in any one or more ways, e.g., crimping, application of adhesive, etc., as will be appreciated by a person skilled in the art. The axial support members 36 can be coupled to an inner surface of the retractor 18, e.g., an inner surface of the inner elongate portion 28, as shown in this exemplary embodiment, and/or the axial support members 36 can be coupled to an outer surface of the retractor 18, e.g., an outer surface of the inner elongate portion 28. The axial support members 36 can be coupled to the inner and/or outer retractor surfaces along any full or partial longitudinal lengths thereof. Alternatively or in addition, as shown in one embodiment in FIG. 2, any one or more axial support members 36′ can be embedded between first and second films 37a, 37b that form an inner elongate portion of a retractor that otherwise can be configured and used similar to the inner elongate portion 28 of the retractor 18. The films 37a, 37b can be configured to provide a protective barrier around the axial support members 36′, thereby helping to prevent the axial support members 36′ from snagging on tissue or other material and from interfering with a surgical instrument inserted through the surgical access device including the films 37a, 37b. The axial support members 36′ located between the films 37a, 37b can also be configured and used similar to the axial support members 36. Although two films 37a, 37b are illustrated in this embodiment, a retractor can include any number of films, e.g., one, four, etc., and axial support members can be located between any of the films and/or on inner and/or outer surfaces of any of the films.

Referring again to FIG. 1, as shown in this embodiment, the housing 12 can be removably coupled via snap-fit to the retractor 18, which as illustrated in this embodiment can be flexible. The housing 12 can be in a fixed position relative to the retractor 18 as shown in this embodiment, or the housing 12 can be movable relative to the retractor 18. Exemplary embodiments of various housings are described in more detail in previously mentioned U.S. Patent Publication No. 2006/0247673 entitled “Multi-port Laparoscopic Access Device” filed Nov. 2, 2006, U.S. patent application Ser. No. 12/399,625 entitled “Methods and Devices for Providing Access to a Body Cavity” filed on Mar. 6, 2009, U.S. patent application Ser. No. 12/399,482 entitled “Methods and Devices for Providing Access to a Body Cavity” filed on Mar. 6, 2009, and U.S. patent application Ser. No. 12/242,765 entitled “Surgical Access Device” filed on Sep. 30, 2008, and in U.S. patent application Ser. No. 12/399,547 entitled “Surgical Access Devices And Methods Providing Seal Movement In Predefined Paths” filed on Mar. 6, 2009, which is hereby incorporated by reference in its entirety.

While any number of sealing ports can be formed in the seal base 14, in this illustrated embodiment, the seal base 14 includes first, second, and third sealing port openings (not shown) formed therein that extend through the seal base 14 in which the first, second, and third sealing ports 16a, 16b, 16c can be seated. In general, the sealing ports 16a, 16b, 16c can define a working channel (not shown) extending therethrough and be configured to receive an instrument therethrough. Each of the sealing ports 16a, 16b, 16c can include a port housing 30a, 30b, 30c, which can be seated directly or indirectly in one of the port openings in the seal base 14, and a sealing element 24a, 24b, 24c, which can be positioned within an associated port housing 30a, 30b, 30c. The port housings 30a, 30b, 30c can each have any shape, height, or angular configuration, but in the embodiment shown in FIG. 1, the port housings 30a, 30b, 30c can each have a cylindrical shape. First, second, and third distal surfaces of the respective port housings 30a, 30b, 30c can be substantially flat such that they can be coplanar with a proximal surface 14a of the seal base 14, as shown. First, second, and third proximal surfaces of the respective port housings 30a, 30b, 30c can likewise be flat, or any one or more can extend at an angle with respect to the proximal surface 14a of the seal base 14, such as described in more detail in previously mentioned U.S. patent application Ser. No. 12/242,765 entitled “Surgical Access Device” filed on Sep. 30, 2008. A sealing element can include at least one instrument seal and/or at least one channel seal, and can generally be configured to contact an instrument inserted through the sealing element's associated sealing port. Exemplary embodiments of various sealing ports are described in more detail in previously mentioned U.S. Patent Publication No. 2006/0247673 entitled “Multi-port Laparoscopic Access Device” filed Nov. 2, 2006, U.S. patent application Ser. No. 12/399,625 entitled “Methods and Devices for Providing Access to a Body Cavity” filed on Mar. 6, 2009, U.S. patent application Ser. No. 12/399,482 entitled “Methods and Devices for Providing Access to a Body Cavity” filed on Mar. 6, 2009, and U.S. patent application Ser. No. 12/242,765 entitled “Surgical Access Device” filed on Sep. 30, 2008.

The sealing ports 16a, 16b, 16c can, as in this illustrated embodiment, each have a central axis that extends substantially perpendicular to the proximal surface 14a of the seal base 14, and the sealing ports 16a, 16b, 16c can each be in a fixed position relative to the housing 12, but any one or more of the sealing ports can be angled relative to the seal base 14 and/or rotatable or otherwise movable relative to the seal base 14 and/or other portion(s) of the housing 12. Additionally or alternatively, any one or more of the sealing ports 16a, 16b, 16c can be configured to be movable relative to any one or more portions of the retractor 18 and/or any others of the sealing ports 16a, 16b, 16c. The sealing ports 16a, 16b, 16c can be attached or mated to the seal base 14 using any attachment or mating mechanism known in the art, but in the illustrated embodiment the sealing ports 16a, 16b, 16c can each mate with the seal base 14 through an interference fit.

The sealing ports 16a, 16b, 16c can each have any size, e.g., working channel diameter configured to allow passage of a surgical instrument having a diameter equal to or less than the working channel diameter. At least two of the sealing ports 16a, 16b, 16c can have different sizes. As shown in this embodiment, the first and second sealing ports 16a, 16b can each have a first diameter D1 configured to allow passage therethrough of a surgical instrument having a diameter equal to or less than the first diameter D1, while the third sealing port 16c can have a second diameter D2 larger than the first diameter D1 and configured to allow passage therethrough of a surgical instrument having a diameter equal to or less than the second diameter D2. The first and second diameters D1, D2 can each have any size, e.g., 10 mm, 12 mm, 7 mm, 3 mm, 5 mm, etc.

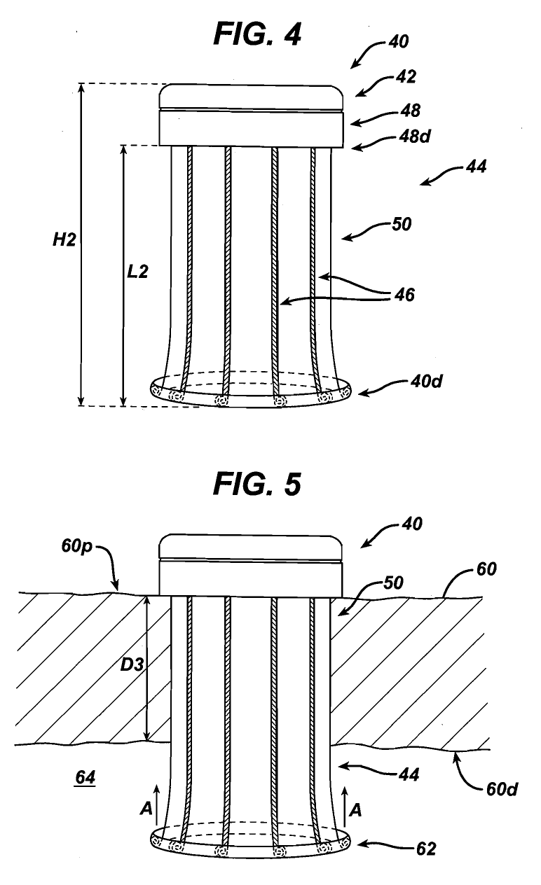

FIGS. 3 and 4 illustrate another embodiment of a surgical access device 40 configured to be positioned within an opening formed in tissue and automatically adjust to a depth of the opening. The device 40 can be configured and used similar to the device 10 discussed above and include a proximal housing 42 and a distal retractor 44 configured to fixedly or removably couple to the housing 42. Similarly, the housing 42 and the retractor 44 can be configured and used similar to the housing 12 and the retractor 18 discussed above, respectively.

In this embodiment, the retractor 44 includes a plurality of axial support members 46 spaced equidistantly around a circumference or perimeter of the retractor 44, although as mentioned above the device 40 can include any number of axial support members 46 arranged in any way. The axial support members 46, e.g., constant force springs, can be configured and used similar to the axial support members 36 discussed above, generally being configured to automatically adjust a longitudinal length of the device 40 when at least a portion of the device 40, e.g., the retractor 44, is positioned in tissue.

The axial support members 46 can be biased to a first, compressed configuration or resting position in which each of the axial support members 46 can be at least partially rolled from distal ends up (in a proximal direction). FIG. 3 shows the device 40 with the axial support members 46 in the first, compressed configuration such that the retractor 44 can be in a first, compressed configuration in which the device 40 has a first longitudinal length or height H1. In the compressed configuration a distal end 40d of the device 40 can be located a first distance L1 from a distal end 48d of a proximal retractor base 48, e.g., with an inner elongate portion 50 of the retractor 44 having a longitudinal length equal to the first distance L1. Although, in some embodiments the first distance L1 can be substantially zero with the axial support members 46 substantially fully rolled such that the distal end 40d of the device 40 can abut the distal end 48d of the proximal retractor base 48. The axial support members 46 can be moved from the first, compressed configuration to a second, expanded configuration, as shown in FIG. 4, in which the axial support members 46 can be extended into a substantially straight position and have a longitudinal length substantially equal to a second distance L2, greater than the first distance L1, between the device's distal end 40d and the proximal retractor base's distal end 48d. Thus, as the axial support members 46 compress, the longitudinal length of the device 40 can decrease. Although the axial support members 46 can be configured to be manually moved from the compressed configuration to the expanded configuration as shown in FIGS. 3 and 4, e.g., by unrolling a rolled portion of the axial support members 46 at a distal end of the retractor 44, the axial support members 46 can additionally or alternatively be configured to be automatically moved from the compressed configuration to the expanded configuration by action of the biasing force exerted by the axial support members 46. The axial support members 46 can be configured to be automatically moved from the compressed configuration to the expanded configuration in a variety of ways, such as by actuating an actuator on a proximal portion of the device 40, e.g., by depressing a button (not shown) or rotating a rotating knob (not shown) located on the housing 42 and coupled to the axial support members 46, as described by way of non-limiting example in U.S. Patent Publication No. 2009/0082731 entitled “Dilating Trocar Cannula” filed Sep. 20, 2007, which is hereby incorporated by reference in its entirety. Similarly, the axial support members 46 can be configured to be manually and/or automatically moved from the expanded configuration to the compressed configuration. A person skilled in the art will appreciate that the axial support members 46 can be configured to automatically adjust the longitudinal length of the device 40 regardless of whether the axial support members 46 are configured to be manually or automatically moved. As discussed further below, a longitudinal length of the axial support members 46, and hence a longitudinal length of the device 40, in the compressed configuration can be defined by a depth of a tissue opening in which the device 40 is positioned.

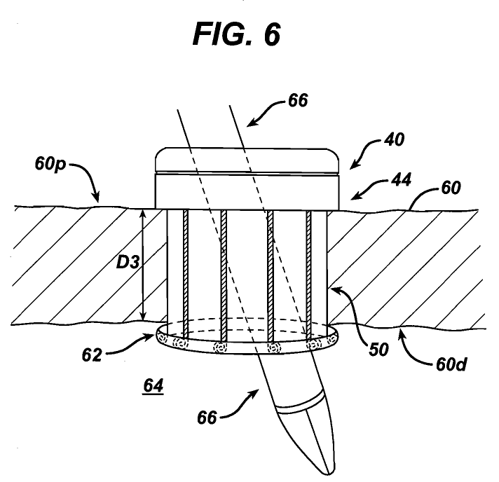

In use, any of the surgical access devices described herein can be positioned within tissue to provide access to a body cavity underlying the tissue. As illustrated in one embodiment in FIGS. 5 and 6, the surgical access device 40 of FIGS. 3 and 4 in use can be positioned within an opening naturally or artificially formed in a tissue 60 in a variety of ways. In one embodiment, the device 40 can be positioned in the tissue 60 fully assembled in the expanded configuration shown in FIG. 5. Being biased to the compressed configuration, prior to positioning the device 40 in the tissue 60 in the expanded configuration, the axial support members 46 can be manually and/or automatically moved to move the device 40 to the expanded configuration. In another embodiment, the device 40 can be positioned partially assembled in the tissue 60 and be fully assembled with a portion of the device 40 positioned in the tissue 60, e.g., the retractor 44 of the device 40 can first be positioned in the tissue 60 and the housing 42 of the device 40 subsequently coupled to the retractor 44. If the tissue 60 and/or the retractor 44 are adequately flexible, the retractor 44 can be angled or pivoted to a desired position to ease attachment of the housing 42 to the retractor 44.

However positioned within the tissue 60, as illustrated in this embodiment in FIG. 5, the retractor 44 as fully assembled can be positioned within an opening or incision formed in the tissue 60, e.g., in the umbilicus, with proximal and distal portions of the retractor 44 positioned on opposed sides of the tissue 60. A proximal portion of the retractor 44 can be positioned on one side of the tissue 60 with a distal surface of the proximal retractor base 48 positioned on and/or proximal to a proximal surface 60p of the tissue 60. A distal flange 62 of the retractor 44 can be positioned any distance distally beyond a distal surface 60d of the tissue 60 in a body cavity 64 underlying the tissue 60. The inner elongate portion 50 of the retractor 44 can thereby be positioned within the tissue 60 with a working channel or passageway (not shown) of the retractor 44 extending through the tissue 60 to provide a path of access to the body cavity 64.

With at least the retractor 44 of the surgical access device 40 positioned in the tissue 60 with the axial support members 46 in the expanded configuration, the axial support members 46 can automatically adjust the longitudinal length of the retractor 44 to approximate a depth D3 of the opening formed in the tissue 60, as discussed above. As illustrated in FIGS. 5 and 6, the axial support members 46 can roll distal end up (in a proximal direction as shown by directional arrows A in FIG. 5) any distance until the distal end 40d of the device 40, e.g., a distal end of the retractor 44, abuts or contacts the distal surface 60d of the tissue 60 at a distal portion of the opening in the tissue 60. The distal surface 60d of the tissue 60 can act as a stop preventing further proximal movement of the axial support members 46, thereby decreasing the height of the retractor 44 to facilitate secure positioning thereof within the tissue 60 with the axial support members 46 exerting a proximally directed force against the distal surface 60d of the tissue 60 adjacent the tissue opening. The distal surface 60d of the tissue 60 can thus define the longitudinal length or height of the retractor 44 in the compressed configuration. In other words, the retractor 44 can move from having the second longitudinal length L2 to having the first longitudinal length L1, with the first longitudinal length L1 being substantially equal to the depth D3 of the tissue opening. Each of the axial support members 46 can proximally roll any distance same or different from any one or more of the other axial support members 46, with the distances varying for any reason, such as because the distal surface 60d of the tissue 60 can be uneven. A person skilled in the art will appreciate that only a portion of a perimeter or circumference of the distal end 40d of the device 40 can abut or contact the distal surface 60d of the tissue 60, e.g., because the distal surface 60d of the tissue 60 can be uneven.

With the surgical access device 40 positioned in the tissue 60 with the axial support members 46 compressed, one or more surgical instruments can be inserted therethrough and into the body cavity 64 where the instruments can help perform any type of surgical procedure. One or more surgical instruments, e.g., a pair of movable jaws 66, can be inserted through the device 40 and into the body cavity 64 through any of the device's one or more sealing ports to help perform at least a portion of a surgical procedure. If the tissue 60 and/or the retractor 44 are adequately flexible, the retractor 44 can be angled or pivoted during use of the device 40 with the movable jaws 66 and/or other surgical tools inserted therethrough. The axial support members 46 can be configured to dynamically adjust the longitudinal length of the retractor 44 by any one or more of the axial support members 46 rolling and/or unrolling any amount as the retractor 44 is angled or pivoted to continuously provide a secure fit of the retractor 44 within the tissue opening. Although a pair of movable jaws 66 are shown inserted through the device 40 in FIG. 6, any surgical device such as a grasper, a scoping device (e.g., an endoscope, a laparoscope, and a colonoscope), a cutting instrument, etc., can be inserted through the device 40. A person skilled in the art will appreciate that the term “grasper” as used herein is intended to encompass any surgical instrument that is configured to grab and/or attach to tissue and thereby manipulate the tissue, e.g., forceps, retractors, movable jaws, magnets, adhesives, stay sutures, etc. A person skilled in the art will also appreciate that the term “cutting instrument” as used herein is intended to encompass any surgical instrument that is configured to cut tissue, e.g., a scalpel, a harmonic scalpel, a blunt dissector, a cautery tool configured to cut tissue, scissors, an endoscopic linear cutter, a surgical stapler, etc.

At any point before, during, or after a surgical procedure, the housing 42 in full or part can be released from the retractor 44, and the retractor 44 can be removed from the tissue 60. With the housing 42 of the device 40 disengaged from the retractor 44, the passageway of the retractor 44 can still provide access to the body cavity 64 underlying the tissue 60. One or more surgical instruments can be advanced through the passageway of the retractor 44, such as a waste removal bag configured to hold waste material, e.g., dissected tissue, excess fluid, etc., from the body cavity 64. The bag can be introduced into the body cavity 64 through the retractor's passageway or other access port. A person skilled in the art will appreciate that one or more surgical instruments can be advanced through the retractor's passageway before and/or after the housing 42 has been attached to the retractor 44.

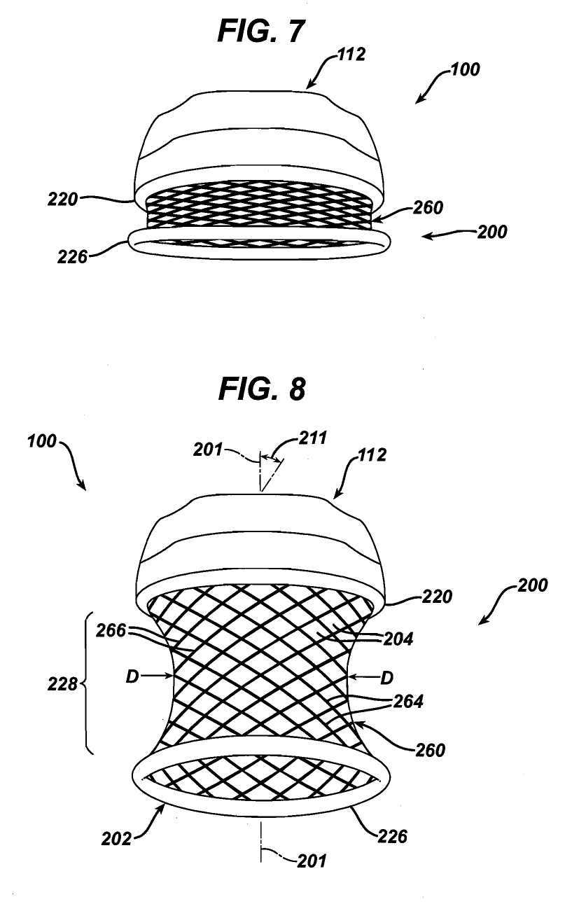

Referring now to FIGS. 7-14, embodiments of surgical devices 100 are illustrated having one or more members, such as one or more elongate members, which extend both axially and circumferentially with respect to a flexible retractor. The surgical devices shown in FIGS. 7-14 may be used as set forth above.

FIGS. 7 and 8 illustrate a surgical device 100 having a seal housing 112 disposed at a proximal end of a flexible tissue retractor 200. In FIG. 7, the retractor 200 is shown in a retracted configuration, and in FIG. 8 the retractor 200 is shown in an extended configuration. The tissue retractor 200 provides an internal passageway 202 extending therethrough, and the retractor is shown including a portion 228 comprising a flexible membrane 204 extending intermediate a proximal flange 220 and a distal flange 226. The proximal flange 220 may comprises a generally circumferentially extending flexible resilient ring, and the distal flange 226 may comprise a generally circumferentially extending flexible resilient ring. The flexible membrane 204 of the retractor can be formed of any suitable material known in the art, e.g., silicone, urethane, thermoplastic elastomer, and rubber.

The portion 228 of retractor 200 is shown including one or more members (indicated generally as structure 260) which extends both axially and circumferentially with respect to the longitudinal axis 201 of the retractor, and which provide a biasing force to urge the retractor from the extended configuration in FIG. 8 to the contracted configuration in FIG. 7. As shown in FIGS. 7 and 8, the portion 228 can have a reduced or “necked down” diameter D when the retractor is in the extended configuration of FIG. 8, as compared to the diameter of the portion 228 when the retractor is in the fully retracted configuration of FIG. 7.

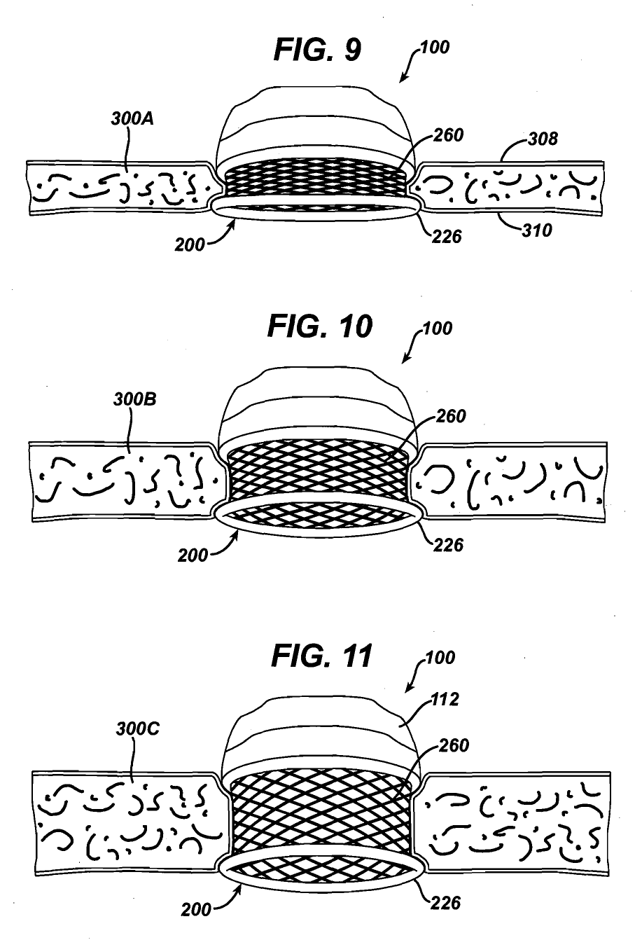

FIGS. 9, 10, and 11 illustrate how the structure 260 resiliently biases the retractor 200 to adjust the axial length of the retractor so that the retractor self adapts to various tissue thicknesses. FIG. 9 illustrates retractor 200 disposed through a relatively thin tissue layer 300A, FIG. 11 illustrates retractor 200 disposed through a relatively thick tissue layer 300C, and FIG. 10 illustrates retractor 200 disposed through an intermediate thickness tissue layer 300B.

In each of the cases shown in FIGS. 9-11, structure 260 may assist in resiliently biasing the proximal flange 220 to fit snugly against an outer skin surface 308 and the distal flange 226 against an inner body cavity surface 310. Additionally, and without being limited by theory, it is believed that the structure 260 can provide circumferentially directed hoop strength resistance to maintain the internal passageway of the flexible membrane of retractor 200 open against the compressive forces of the tissue/incision which might otherwise cause the retractor 200 to be pinched or otherwise pressed closed or partially closed by the surrounding tissue layer.

The structure 260 may be disposed on an inner surface of membrane 204, an outer surface of membrane 204, within membrane 204 (e.g. such as between layers of membrane 204 or by being molded inside membrane 204), or combinations thereof. In FIGS. 7 and 8, the structure 260 is shown comprising a plurality of elongate members 264 and 266 operatively associated with the flexible membrane 204, such as by being fastened to, molded with, or otherwise joined to flexible membrane 204. The members 264 and 266 may also be joined to or otherwise associated to the flanges 220 and 226. For instance, the members 264 and 266 may have ends which are fastened or otherwise joined to flexible o-rings of flanges 220 and 226.

The members 264 extend both axially and circumferentially, and are shown as being generally parallel and spaced apart with respect to each adjacent other member 264. Likewise, member 266 are shown as being generally parallel to and spaced apart from each adjacent other member 266. The members 264 are shown inclined with respect to axis 201 and members 266, and members 266 are shown inclined with respect to axis 201 and members 264. In one embodiment members 264 may be inclined with respect to the axis 201 at an angle 211 of between 20 degrees and about 70 degrees, more particularly between about 30 degrees and about 60 degrees. In one embodiment, members 264 may be inclined with respect to members 266 at an angle 213 (FIG. 13) of between about 20 degrees and about 160 degrees, and more particularly, between about 45 degrees and about 135 degrees. The members 264/266 may also be sized and shaped to grip or engage the side of the tissue incision in which the retractor is inserted to assist in retaining the retractor in the incision.

The structure comprising members 264 and 266 may be metallic (e.g. a shape memory alloy such as Nitinol), non-metallic, semi-metallic, or combinations thereof. The members may be magnetic or nonmagnetic. Where the members are metallic, they may be ferrous or non-ferrous. For instance, the members may comprise iron, aluminum, spring steel, stainless steel. Various suitable materials include Cold-Rolled Spring Steel Blue Tempered or Annealed SAE 1074, Cold-Rolled Spring Steel Blue Tempered or Annealed SAE 1064, Cold-Rolled Spring Steel Blue Tempered or Annealed SAE 1070, Cold-Rolled Spring Steel Blue Tempered Clock Steel SAE 1095, Chromium Vanadium ASTM A231, Chromium Silicon ASTM A401, Stainless Steel type 302 ASTM A313, Stainless Steel Type 304 ASTM A313, Stainless Steel type 316 ASTM A313, Stainless Steel Type 17-7 PH ASTM A313, Stainless Steel Type 414 SAE 51414, Stainless Steel Type 420 SAE 51420, Stainless Steel Type 431 SAE 51431.

The members 264 and 266 may have an elongate form, and may be in any suitable form, such as in the form of a wire, strand, filament (monofilament or polyfilament), or combinations thereof. The members 264 and 266 may be formed separately from flexible membrane 204, or alternatively, they may be in the form of ribs or projections integrally formed with membrane 204, such as by molding, casting, or the like. In one embodiment, the members 264 and 266 are formed of a material that is different from and stiffer than that from which membrane 204 is formed. Alternatively, the members 264 and 266 can be formed of the same material from which membrane 204 is formed.

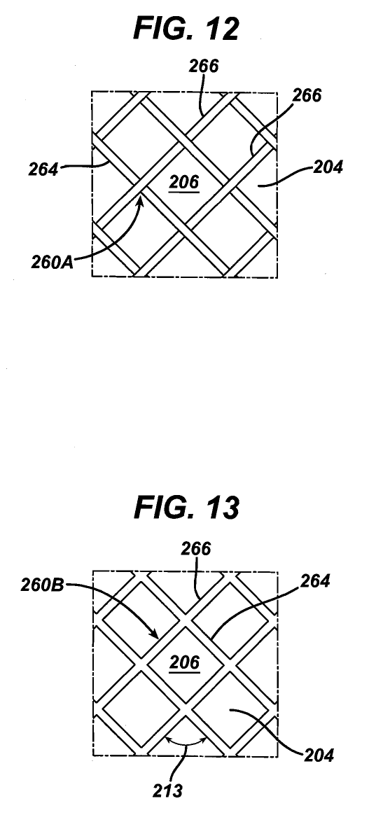

Referring to FIG. 12, in one embodiment, members 264 and 266 may be woven together to form a woven network 260A which may be joined to the inner or outer surface of membrane 204, or disposed within layers of membrane 204. In another embodiment, such as shown in FIG. 13, members 264 and 266 may together provide a nonwoven structure 260B. In both embodiments shown in FIGS. 12 and 13, the structure 260 can provide window like enclosed regions 206 of the flexible membrane 204 defined by the intersection of members 264 and 266.

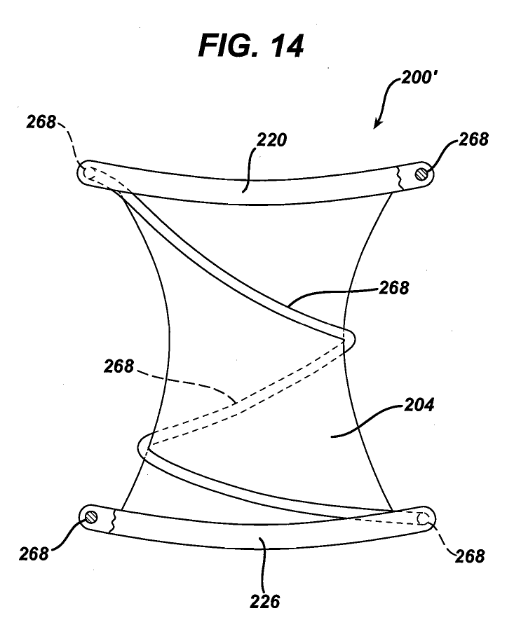

Referring to FIG. 14, a flexible retractor 200′ is illustrated having a flexible membrane 204 extending from proximal flange 220 to distal flange 226, and having a generally helical coil-like elongate member 268. Member 268 may extend around the full circumference of the retractor 200 one or more times, and in FIG. 14, the member 268 is shown in phantom cutaway as extending into and around the circumference of flanges 220 and 226. The member 268 can provide a resilient ring structure extending circumferentially within the flanges 220, 226. The member 268 can be formed of a metallic or non-metallic wire, strand, or filament, and in one embodiment may comprise a non-metallica elastomeric material.

In FIG. 14, the member 268 extends around the membrane 204 in a counter clockwise manner from proximal flange 220 to distal flange 226 as viewed from the proximal end of retractor 200′. If desired, a second member can be provided extending in a generally clockwise fashion. In yet another embodiment, a single wire, strand, or filament can be employed to extend around the circumference of flange 220, then axially and circumferentially from flange 220 to flange 226 (such as in a clockwise manner), then around flange 226, then axially and circumferentially back to flange 220 (such as in a counter clockwise manner).

As will be appreciated by those skilled in the art, any and all of the embodiments disclosed herein can be interchangeable with one another as needed. For example, an exemplary surgical access device kit could include multiple housings and seal bases with one or more retractors. Each seal base and housing combination can have different quantities and/or sizes of sealing ports enabling various combinations of variously sized surgical instruments to be inserted therethrough as needed in particular application. Various release mechanism known in the art can be used to releasably attach the various base members and housings to a retractor.

There are various features that can optionally be included with any and all of the surgical access device embodiments disclosed herein. For example, a component of the device, such as a seal base, housing, retractor, etc., can have one or more lights formed thereon or around a circumference thereof to enable better visualization when inserted within a patient. As will be appreciated, any wavelength of light can be used for various applications, whether visible or invisible. Any number of ports can also be included on and/or through the surgical access devices to enable the use of various surgical techniques and devices as needed in a particular procedure. For example, openings and ports can allow for the introduction of pressurized gases, vacuum systems, energy sources such as radiofrequency and ultrasound, irrigation, imaging, etc. As will be appreciated by those skilled in the art, any of these techniques and devices can be removably attachable to the surgical access device and can be exchanged and manipulated as needed.

The embodiments described herein can be used in any known and future surgical procedures and methods, as will be appreciated by those skilled in the art. For example, any of the embodiments described herein can be used in performing a sleeve gastrectomy and/or a gastroplasty, as described in U.S. application Ser. No. 12/242,765 entitled “Surgical Access Device” filed on Sep. 30, 2008; U.S. application Ser. No. 12/242,711 entitled “Surgical Access Device with Protective Element” filed on Sep. 30, 2008; U.S. application Ser. No. 12/242,721 entitled “Multiple Port Surgical Access Device” filed on Sep. 30, 2008; U.S. application Ser. No. 12/242,726 entitled “Variable Surgical Access Device” filed on Sep. 30, 2008; U.S. application Ser. No. 12/242,333 entitled “Methods and Devices for Performing Gastrectomies and Gastroplasties” filed on Sep. 30, 2008; U.S. application Ser. No. 12/242,353 entitled “Methods and Devices for Performing Gastrectomies and Gastroplasties” filed on Sep. 30, 2008; and U.S. application Ser. No. 12/242,381 entitled “Methods and Devices for Performing Gastroplasties Using a Multiple Port Access Device” filed on Sep. 30, 2008, all of which are hereby incorporated by reference in their entireties.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination, e.g., a seal base, a housing, a proximal retractor base, etc. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety

What is claimed is:

1. A flexible surgical retractor comprising:
a flexible membrane providing an access passageway therethrough; and
a structure extending circumferentially and axially with respect to the membrane.

2. The flexible retractor of claim 1 wherein the structure provides axial contraction of the membrane.

3. The flexible retractor of claim 1 wherein the structure provides circumferential hoop strength to the flexible membrane.

4. The flexible retractor of claim 1 wherein the structure comprises a woven structure.

5. The flexible retractor of claim 1 wherein the structure comprises a non-woven structure.

6. The flexible retractor of claim 1 wherein the structure comprises a non-metallic material.

7. The flexible retractor of claim 1 wherein the structure comprises a shape memory material.

8. The flexible retractor of claim 1 wherein the structure extends at least once around the circumference of the retractor.

9. A surgical device, comprising:
a housing having multiple instrument opening; and
a retractor having an inner passageway extending therethrough, the retractor configured to be releasably coupled to the housing; wherein the retractor comprises:
a flexible membrane; and
a structure extending circumferentially around the flexible membrane, the structure operative for adjusting the axial length of the retractor.

10. The device of claim 9 wherein the structure is operative to axially contract the length of the retractor.

11. The device of claim 9 wherein the structure comprises a woven structure.

12. The device of claim 9 wherein the structure comprises a non-woven structure.

13. The device of claim 9 wherein the structure imparts circumferential hoop strength to the at least a portion of the retractor intermediate a proximal end of the retractor and a distal end of the retractor.

14. The device of claim 9 wherein the flexible membrane extends from a proximal flange to a distal flange, and wherein the structure extends circumferentially and axially with respect to the flexible membrane.

15. The device of claim 9, wherein the retractor is configured to move from the first height to the second height when the retractor is positioned in an opening in tissue, the retractor being configured when at the second height to abut tissue at a distal portion of an opening in tissue.

16. The device of claim 0, wherein the retractor in a resting position has the second height.

17. The device of claim 9 comprising at least one constant force spring.

18. The device of claim 1 wherein the structure comprises a ferrous material.

19. The device of claim 1 wherein the structure comprises a non-ferrous material.

20. The device of claim 1 wherein the structure comprises spring steel.
